(19) United States
(12) Patent Application Publication
(10) Pub. No.: US 2008/0306277 A1
(43) Pub. Date: Dec. 11, 2008

(54) Process for Preparation of Chiral Amlodipine Gentisate

(76) Inventors: Jae-Sun Kim, Gyeonggi-do (KR); Nam Ho Kim, Gyeonggi-do (KR); Nam Kyu Lee, Gyeonggi-do (KR)

Correspondence Address: FROMMER LAWRENCE & HAUG, 745 FIFTH AVENUE- 10TH FL., NEW YORK, NY, 10151, US

(21) Appl. No.: 12/090,264

(22) PCT Filed: Oct. 17, 2006
(86) PCT No.: PCT/KR2006/004206

§ 371 Date: Aug. 21, 2008

(30) Foreign Application Priority Data
Oct. 17, 2005 (KR) 10-2005-0097532

(51) Int. Cl. C07D 211/90

(52) U.S. Cl. 546/321

(57) ABSTRACT

The present invention relates to a process for the preparation of optically pure amlodipine gentisate, more particularly to a continuous process for the preparation of optically pure amlodipine gentisate with good yield and high optical purity. The processes can be preformed by first reacting racemic (R,S)-amlodipine and optically pure O,O′-dibenzoyltartaric acid in the presence of a solvent including isopropanol to prepare (R)- or (S)-amlodipine dibenzoyltartarate diastereomer or a solvate thereof, treating the prepared amlodipine diastereomeric salt or a solvate thereof with a base and then finally adding gentisic acid.

### TECHNICAL FIELD

The present invention relates to a process for the preparation of optically pure amlodipine gentisate, more particularly to a continuous process for the preparation of optically pure amlodipine gentisate with good yield and high optical purity. The processes can be preformed by first reacting racemic (R,S)-amlodipine and optically pure O,O′-dibenzoyltartaric acid in the presence of a solvent including isopropanol to prepare (R)- or (S)-amlodipine dibenzoyltartarate diastereomer or a solvate thereof, treating the prepared amlodipine diastereomeric salt or a solvate thereof with a base and then finally adding gentisic acid.

### BACKGROUND ART

Amlodipine is the common name of the compound represented by the formula (1) below having the chemical name of 3-ethyl-5-methyl-2-(2-aminoethyoxymethyl)-4-(2-chlorophenyl)-6-methyl-1,4-dihydr o-3,5-pyridine dicarboxylate:

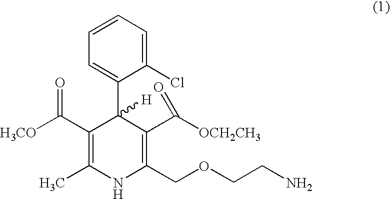

As one of calcium channel blockers, amlodipine is used to treat ischemic heart diseases and hypertension. It is known to be a useful and effective substance with long-term activity.

As seen in the formula (1) above, amlodipine is a chiral compound having a chiral center. In general, pure stereoisomers have better therapeutic effect than racemic mixtures. And, the chiral compounds tend to have different pharmacological properties, depending on the steric arrangement of the isomer compounds or their salts. It is known that the (S)-(−)-isomer of amlodipine is a potent calcium channel blocker and the (R)-(+)-isomer is effective in treating or protecting atherosclerosis. Accordingly, there is a need for the development of a technique to isolate such chiral compounds as amlodipine into optically pure isomers.

Amlodipine was first reported as one of novel 1,4-dihydropyridines in European Patent Publication No. 89,167. European Patent Publication No. 89,167 discloses an acid adduct as an example of pharmaceutically acceptable salts of 1,4-dihydropyridine. The pharmaceutically acceptable acid adduct is formed from an acid that forms a nontoxic acid adduct including a pharmaceutically acceptable anion, including hydrochloride, hydrobromide, sulfate, phosphate, acetate, malate, fumarate, lactate, tartrate, citrate and gluconate. Of them, malate is most preferable.

A free base form of amlodipine is desirable from a pharmaceutical point of view, but an acid adduct with a pharmaceutically acceptable acid is utilized because of its poor stability.

Korean Patent No. 90,479 describes that, in preparing a pharmaceutically acceptable salt, the four physical and chemical standards of (1) superior solubility, (2) superior stability, (3) non-hygroscopicity and (4) processability into a tablet form should be satisfied. It is very difficult to find a pharmaceutically acceptable acid adduct that satisfied all the four standards. In fact, even the malate salt which is preferred as the most preferable pharmaceutical form has a stability problem since it tends to be disintegrated within weeks in a solution.

Korean Patent No. 91,020 discloses a benzenesulfonate salt (hereinafter referred to as “besylate”) as an acid adduct having superior stability. Amlodipine besylate has various advantages over other known amlodipine salts and is known to have characteristics suitable for the preparation of pharmaceutical forms. However, according to the researches performed by the present inventors, the amlodipine besylate offers superior physicochemical properties when the slat is formed with a racemic amlodipine base, but it does not show good physicochemical properties when it is formed as a pure isomer of amlodipine.

Therefore, the present inventors have studied gentisate, an ideal acid adduct for the pharmaceutically acceptable salt of amlodipine in a pure isomer form. In addition, they have made intensive researches to develop an economical and efficient method for preparing (R)-amlodipine gentisate or (S)-amlodipine gentisate.

Most of the recent commercial techniques for resolving the isomers of amlodipine are based on forming diastereomeric salts of amlodipine using D- or L-tartaric acid and isolating them using an appropriate solvent. The use of the diastereomeric salts of amlodipine can be an effective way of resolving the isomers because they can be physically isolated and they can be easily neutralized using a base.

For example, U.S. Pat. No. 6,046,338 discloses a method of isolating the optical isomers of amlodipine by forming the salts of tartaric acid in the presence of dimethyl sulfoxide (DMSO). U.S. Pat. No. 6,646,131 discloses a method of isolating tartaric acid salts using deuterium-substituted dimethyl sulfoxide (DMSO-d₆). Further, U.S. Patent Publication No. 0130321 discloses a method of isolating the optical isomers of amlodipine by forming the salts of tartaric acid in the presence of dimethylacetamide.

The aforementioned isolation methods disclose ways to produce amlodipine isomers with a relatively high optical purity. However, since these methods use solvents such as dimethyl sulfoxide, deuterium-substituted dimethyl sulfoxide or dimethylacetamide, which are expensive and difficult to re-collect and tend to remain due to their relatively high boiling points, and thus they are largely limited with respect to their processing and economical point of view.

The present inventors have found that amlodipine gentisate has low toxicity, sufficient stability and improved medicinal effect and remains within the effective blood level for a long period of time after administration, making it an effective drug for treating hypertension and other cardiovascular diseases, and thus have filed a patent application regarding amlodipine gentisate and a process for preparing the same [Korean Patent Application No. 2004-100613]. Korean Patent Application No. 2004-100613 relates to a method of obtaining an amlodipine gentisate optical isomer or a racemate thereof by reacting an (R)- or (S)-amlodipine isomer or a racemate thereof with gentisic acid to obtain an amlodipine gentisate isomer or a racemate thereof.

The present invention aims at a method enabling a commercial scale production of the optically pure (R)- or (S)-amlodipine gentisate.

The present inventors have researched to develop a process for directly preparing optically pure (R)- or (S)-amlodipine gentisate from a free base form of (R,S)-amlodipine.

In doing so, they found out that the amlodipine dibenzoyltartrate diastereomeric isomers produced by reacting racemic (R,S)-amlodipine with optically pure O,O′-dibenzoyltartaric acid have large solubility differences in a solvent including isopropanol and, thus, can be effectively isolated from each other by taking advantage of the solubility difference. Further, the present inventors have developed a simple, continuous one-step process of obtaining (S)- or (R)-amlodipine gentisate from the optically isolated (R)- or (S)-amlodipine dibenzoyltartrate, and thus completed the present invention.

### Disclosure of the Invention

It is an object of the present invention to provide a process for the preparation of optically pure (R)- or (S)-amlodipine gentisate from racemic (R,S)-amlodipine, which is applicable to commercial-scale production.

The preparation process of optically pure (R)- or (S)-amlodipine gentisate in accordance with the present invention comprises the steps of:

a) preparing a diastereomeric mixture of amlodipine dibenzoyltartrate from (R,S)-amlodipine using isopropanol as a solvent and optically pure O,O′-dibenzoyltartaric acid, and then optically isolating the same; and

b) treating the isolated amlodipine dibenzoyltartrate diastereomer with a base, and subsequently obtaining optically pure amlodipine gentisate by adding gentisic acid to the resulting free form in a single continuous step.

Hereunder is given a more detailed description of the present invention.

The present invention relates to a process for preparing optically pure (R)- or (S)-amlodipine gentisate directly from an (R,S)-amlodipine racemate.

That is, the present invention relates to a preparation process of optically pure (R)- or (S)-amlodipine gentisate from an (R,S)-amlodipine racemate, wherein the starting material is reacted with optically pure O,O′-dibenzoyltartaric acid to prepare a diastereomeric mixture of of amlodipine dibenzoyltartrate, which is optically isolated by taking advantage of the difference in solubility of the isomers in an isopropanol solvent and the isolated chiral amlodipine dibenzoyltartrate is treated with a base and followed by gentisic acid to obtain the targeted optically pure salts, (R)- or (S)-amlodipine gentisate.

The present invention is characterized in that, for the resolution of racemic (R,S)-amlodipine, isopropanol is used as a reaction solvent and optically pure O,O′-dibenzoyltartaric acid is selectively utilized as a resolving agent. Isopropanol, which is used as solvent in the present invention, is much less expensive than dimethyl sulfoxide, deuterium-substituted dimethyl sulfoxide or dimethylacetamide, which have been usually utilized for the optical isolation of amlodipine, leaves little residues after reaction because of low boiling point, and is also advantageous in re-collection and purification, thereby simplifying the post-treatment process. Optically pure O,O′-dibenzoyltartaric acid, which is selectively used as a resolving agent in the present invention, is a chiral compound with two benzoyl groups in tartaric acid. When compared with optically pure tartaric acid, which has been usually utilized for the optical isolation of amlodipine, the diastereomeric salts of O,O′-dibenzoyltartaric acid show significantly increased solubility in an isopropanol solvent. Therefore, the two diastereomeric salts can be easily isolated from each other by taking advantage of the solubility difference without using such an expensive solvent as dimethyl sulfoxide.

Hereunder is given a more detailed description of the process for the preparation of optically pure amlodipine gentisate in accordance with the present invention, and centered upon the selection of the optical resolving agent.

The following Scheme 1 describes the process of preparing (R)-(+)-amlodipine gentisate selectively using O,O′-dibenzoyl-L-tartaric acid as a resolving agent.

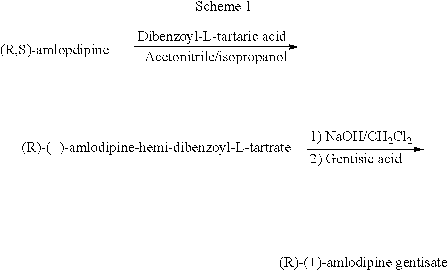

The preparation process in accordance with Scheme 1 comprises the steps of:

a) reacting racemic (R,S)-amlodipine with O,O′-dibenzoyl-L-tartaric acid in a solvent including isopropanol to prepare (R)-(+)-amlodipine-hemi-dibenzoyl-L-tartrate or a solvate thereof, and then optically isolating the same; and

b) treating the isolated chiral amlodipine dibenzoyltartrate or the solvate thereof with a base and followed by gentisic acid to prepare (R)-(+)-amlodipine gentisate through a continuous single process.

The following Scheme 2 describes the process of preparing (S)-(-)-amlodipine gentisate selectively using O,O′-dibenzoyl-D-tartaric acid as a resolving agent.

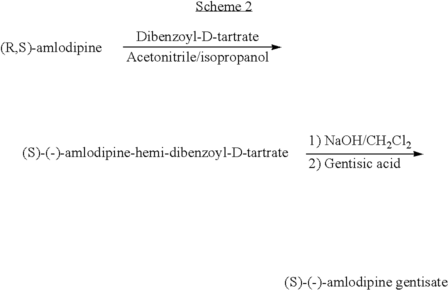

The preparation process in accordance with Scheme 2 comprises the steps of:

a) reacting racemic (R,S)-amlodipine with O,O′-dibenzoyl-D-tartaric acid in a isopropanol solvent to prepare (S)-(−)-amlodipine-hemi-dibenzoyl-D-tartrate or a solvate thereof, and then optically isolating the same; and

b) treating the isolated chiral amlodipine dibenzoyltartrate or the solvate thereof with a base and followed by gentisic acid to prepare (S)-(−)-amlodipine gentisate through a continuous single process.

In the preparation process in accordance with Scheme 1 or 2, the chiral amlodipine existing in the remainder of the chiral amlodipine dibenzoyltartrate or the solvate thereof remaining after the re-collection may be isolated and recovered.

In the preparation process in accordance with the present invention, the optical resolving agent O,O′-dibenzoyl-L-tartaric acid or O,O′-dibenzoyl-D-tartaric acid is used within 0.2-0.6 mole per 1 mole of (R,S)-amlodipine. If the agent is used outside the above range, it is difficult to maximize the yield and optical purity of the resultant chiral salt.

The isopropanol solvent used as a reaction solvent in the present invention may be either a pure isopropanol or a mixed solvent comprising isopropanol as main solvent and an appropriate cosolvent. The cosolvent mixed with the isopropanol is selected from water, ketones, alcohols, ethers, amides, esters, hydrocarbons, chlorohydrocarbons and nitrites. Preferred examples of ketones include acetone and methyl ethyl ketone (MEK). Preferred examples of alcohols include C₁-C₇ saturated alcohols such as isopropanol. Preferred examples of ethers include diethyl ether and tetrahydrofuran (THF). Preferred examples of amides include N,N-dimethylformamide (DMF), N,N-dimethylacetamide (DMAc) and N,N′-dimethylpropyleneurea (DMPU). Preferred examples of esters include acetates such as ethyl acetate (EtOAc). Preferred examples of hydrocarbons include C₅-C₁₀ hydrocarbons such as toluene. Preferred examples of chlorohydrocarbons include chloroform, dichloromethane, 1,2-dichloroethane and 1,1,1-trichloroethane. Preferred examples of nitriles include C₂-C₇ nitriles such as acetonitrile. More specifically, the cosolvent that can be used in the present invention is selected, for example, from water, acetone, acetonitrile, dimethyl sulfoxide, dimethylacetamide, methyl ethyl ketone, tetrahydrofuran, ethyl acetate, dichloromethane, dimethylformamide, toluene, methanol, ethanol, t-butanol and N,N′-dimethylpropyleneurea. The maximum content of the cosolvent used along with isopropanol, the main solvent, depends on the particular cosolvent used. Those skilled in the art may easily determine the appropriate content for obtaining a precipitate for each case. Preferably, the cosolvent is used in less than 50 vol % per 100 vol % of isopropanol, the main solvent. If the cosolvent is used in excess of 50 vol % per 100 vol % of isopropanol, the solubility difference between the amlodipine salt diastereomeric isomers becomes small, thereby significantly reducing the optical purity.

In the preparation process in accordance with the present invention, amlodipine dibenzoyltartrate or the solvate thereof is obtained as a precipitate.

Isolation and collection of the chiral amlodipine salts from the reaction solution can be performed by the methods well known by those skilled in the art. For example, filtration, centrifugation, decantation, etc., may be applied. Of them, filtration or centrifugation is preferable, and filtration is more preferable. As well-known by those skilled in the art, an isolation technique applicable to a single optical isomer may be applied to the isolation of other optical isomer.

The isolated and collected diastereomeric amlodipine salt or the solvate thereof is treated with a base, and gentisic acid is added to prepare the desired gentisate salt of the amlodipine optical isomer.

The base is selected from a hydroxide, an oxide, a carbonate, a bicarbonate and an amide of an alkali metal or an alkaline earth metal. Preferably, alkali metal hydroxide or oxide is used. Particularly preferably, sodium hydroxide is used. When treated with the base, the diastereomeric amlodipine salt is converted into a free base form. The reaction solution including the free base form of amlodipine is extracted using an organic solvent and concentrated. Then, the following crystallization is performed by adding hexane, etc., and gentisic acid is finally added to prepare the gentisate salt of amlodipine optical isomer.

The present invention also relates to a preparation process in which, after the extraction of the reaction solution including the amlodipine isomer with the organic solvent, gentisic acid is added without concentrating the organic solvent to obtain the gentisate salt of the desired amlodipine isomer through crystallization. That is, the salts of (R)- or (S)-amlodipine gentisate in the organic solvent are salted out as crystallized by the solubility difference, thus enhancing the optical purity of the final product. Since the processes of concentrating and treating with such solvent as hexane to obtain the amlodipine isomer base can be omitted, it is very useful in a commercial scale production. To obtain the amlodipine isomer base as intermediate and then react it with gentisic acid, the extraction solution should be concentrated as much as possible, and heated and dried under reduced pressure after crystallization by adding hexane. Subsequently, the process of dissolving the obtained amlodipine isomer base in a solvent, adding gentisic acid and performing filtration shall be followed. Such a two-step process is disadvantageous in terms of solvent consumption, time, labor force and production yield. For a commercial-scale production, a one-step process as proposed by the present invention is desirable.

For the organic solvent used in the extraction, the one in which amlodipine gentisate has a low solubility is preferable. Various solvents may be used, but dichloromethane is preferable.

Gentisic acid may be added in solid form or as dissolved in a solvent. Preferably, gentisic acid is used within 0.1-5.0 equivalents of amlodipine, from the economical point of view.

In accordance with the optical isolation process of the present invention, optically active amlodipine salts with high optical purity of 98-100% e.e. can be obtained efficiently.

### BEST MODE FOR CARRYING OUT THE INVENTION

Practical and preferred embodiments of the present invention are illustrated as shown in the following examples. However, it will be appreciated that those skilled in the art may, in consideration of this disclosure, make modifications and improvements within the spirit and scope of the present invention.

Optical purity of the compounds prepared in the examples was measured by chiral HPLC. The HPLC condition for isolation was as follows:

Column: Ultron ES-OVM (Ovomucoid), 150 mm×4.6 I.D, 5 μm

Flow rate: 1 mL/min

Detection wavelength: 237.4 nm

Eluent: Dibasic sodium phosphate buffer (20 mM, pH 7)/acetonitrile (80/20, v/v)

Sample: Dissolved in acetonitrile at 0.1 mg/mL, added in 10 μL or 5 μL

### EXAMPLE 1

### Preparation of (S)-(−)-Amlodipine Gentisate from (R,S)-Amlodipine

1) Preparation of (S)-(−)-amlodipine-hemi-dibenzoyl-D-tartrate

163.6 g of (R,S)-amlodipine was dissolved in a 3 L of an acetonitrile/isopropanol (1/9) mixed solution and stirred while heating it at 55° C. 35.8 g (0.25 molar equivalent) of dibenzoyl-D-tartaric acid dissolved in 1 L of an acetonitrile/isopropanol (1/9, v/v) mixed solution was added and stirring was performed for 10 more minutes. Separately prepared 0.2 g of (S)-(−)-amlodipine-hemi-dibenzoyl-D-tartrate (>99.5% d.e.) was added and stirring was performed for 3 hours at room temperature. The resulting solid substance was filtered and collected, washed with 500 mL of an acetonitrile/isopropanol (1/9, v/v) mixed solution and dried under vacuum at 50° C. overnight to obtain 98.2 g (theoretical yield: 83.5%) of (S)-(−)-amlodipine-hemi-dibenzoyl-D-tartrate.

Melting point: 116-118° C.; elemental analysis of C₂₀H₂₅N₂O₅Cl 0.5[C₁₈H₁₄O₈]: C 59.10%, H 5.51%, N 4.63%; theoretical: C 59.23%, H 5.49%, N 4.76%; chiral HPLC: 99.0% d.e.

2) Preparation of (S)-(−)-Amlodipine Gentisate

5.88 g of the (S)-(−)-amlodipine-hemi-dibenzoyl-D-tartrate obtained in 1) of Example 1 was stirred in a mixed solution of 56 mL of CH₂Cl₂ and 56 mL of 2 N NaOH (aqueous solution) for 30 minutes. Subsequently, the organic solution was separated and washed once with water. The organic layer was filtered with a filter paper, 1.54 g of gentisic acid dissolved in 5 mL of acetone was added and stirring was performed for 2 hours at room temperature. The resulting solid substance was filtered and collected and dried under vacuum at 50° C. overnight to obtain 5.18 g (92%) of (S)-(−)-amlodipine gentisate.

Melting point: 162-165° C.; elemental analysis of C₆H₃₁N₂O₉Cl: C 57.40%, H 5.60%, N 4.80%; theoretical: C 57.60%, H 5.55%, N 4.98%; chiral HPLC: 99.5% e.e.

### EXAMPLE 2

### Preparation of (R)-(+)-Amlodipine Gentisate from (R,S)-Amlodipine

1) Preparation of (R)-(+)-amlodipine-hemi-dibenzoyl-L-tartrate

163.6 g of (R,S)-amlodipine was dissolved in 3 L of an acetonitrile/isopropanol (1/9) mixed solution and stirred while heating it at 55° C. 35.8 g (0.25 molar equivalent) of dibenzoyl-L-tartaric acid dissolved in 1 L of an acetonitrile/isopropanol (1/9, v/v) mixed solution was added and stirring was performed for 10 more minutes. Separately prepared 0.2 g of (R)-(+)-amlodipine-hemi-dibenzoyl-L-tartrate (>99.5% d.e.) was added and stirred for 3 hours at room temperature. The resulting solid substance was filtered and collected, washed with 500 mL of an acetonitrile/isopropanol (1/9, v/v) mixed solution and dried under vacuum at 50° C. overnight to obtain 97.0 g (theoretical yield: 82%) of (R)-(+)-amlodipine-hemi-dibenzoyl-L-tartrate.

Melting point: 115-117° C.; elemental analysis Of C₂₀H₂₅N₂O₅Cl 0.5[C₈H₁₄O₈]: C 59.15%, H 5.63%, N 4.66%; theoretical: C 59.23%, H 5.49%, N 4.76%; chiral HPLC: 98.4% d.e.

2) Preparation of (R)-(+)-amlodipine gentisate

5.88 g of the (R)-(+)-amlodipine-hemi-dibenzoyl-L-tartrate obtained in 1) of Example 2 was stirred in a mixed solution of 56 mL of CH₂Cl₂ and 56 mL of 2 N NaOH (aqueous solution) for 30 minutes. Subsequently, the organic solution was separated and washed once with water. The organic layer was filtered with a filter paper, 1.54 g of gentisic acid dissolved in 5 mL of acetone was added and stirring was performed for 2 hours at room temperature. The resulting solid substance was filtered and collected and dried under vacuum at 50° C. overnight to obtain 4.95 g (88%) of (R)-(+)-amlodipine gentisate.

Melting point: 161-164° C.; elemental analysis of C₆H₃₁N₂O₉Cl: C 57.44%, H 5.62%, N 4.83%; theoretical: C 57.60%, H 5.55%, N 4.98%; chiral HPLC: 99.0% e.e.

### INDUSTRIAL APPLICABILITY

As apparent from the above description, the present invention enables an efficient isolation of (R,S)-amlodipine optical isomers utilizing the difference in solubility of the diastereomeric amlodipine salts in an isopropanol solvent having a low boiling point and using dibenzoyl-L-tartaric acid or dibenzoyl-D-tartaric acid as an optical resolving agent. In particular, the present invention can be usefully applied in the industry after the treatment of the amlodipine dibenzoyltartarate diastereomeric salts obtained as a reaction intermediate with a base and the extraction using an organic solvent because the optically pure amlodipine gentisate salts can be obtained directly by adding gentisic acid without the need of additional concentration or a complex treatment processes.

Those skilled in the art will appreciate that the concepts and specific embodiments disclosed in the foregoing description may be readily utilized as a basis for carrying out the same purposes of the present invention. Those skilled in the art will also appreciate that such equivalent embodiments do not depart from the spirit and scope of the present invention as set forth in the appended claims.

1. A process for the preparation of optically pure (R)- or (S)-amlodipine gentisate comprising the steps of:
a) preparing a diastereomeric mixture of amlodipine dibenzoyltartrate from racemic (R,S)-amlodipine using isopropanol as a solvent and optically pure O,O′-dibenzoyltartaric acid, and then optically isolating the same; and
b) treating the isolated amlodipine dibenzoyltartrate diastereomer with a base and subsequently obtaining optically pure amlodipine gentisate by adding gentisic acid to the resulting free form in a single continuous step.

2. The preparation process as set forth in claim 1, which comprises the steps of:
a) preparing (R)-(+)-amlodipine-hemi-dibenzoyl-L-tartrate or a solvate thereof from racemic (R,S)-amlodipine using isopropanol as a solvent and O,O′-dibenzoyl-L-tartaric acid, and
b) treating the isolated amlodipine dibenzoyltartrate diastereomer or the solvate thereof with a base and subsequently obtaining (R)-(+)-amlodipine gentisate by adding gentisic acid to the resulting free form in a single continuous step.

3. The preparation process as set forth in claim 1, which comprises the steps of:
a) preparing (s)-(−)-amlodipine-hemi-dibenzoyl-D-tartrate or a solvate thereof from (R,S)-amlodipine using isopropanol as a solvent and O,O′-dibenzoyl-D-tartaric acid, and
b) treating the isolated amlodipine dibenzoyltartrate diastereomer or the solvate thereof with a base and subsequently obtaining (S)-(−)-amlodipine gentisate by adding gentisic acid to the resulting free form in a single continuous step.

4. The preparation process as set forth in claim 1, wherein the chiral O,OΔ-dibenzoyltartaric acid is used within 0.2-0.6 mole per 1 mole of the racemic (R,S)-amlodipine.

5. The preparation process as set forth in claim 1, wherein the isopropanol solvent is isopropanol or a mixed solvent of isopropanol and a cosolvent selected from water, ketones, alcohols, ethers, amides, esters, hydrocarbons, chlorohydrocarbons and nitrites.

6. The preparation process as set forth in claim 5, wherein the cosolvent is selected from water, acetone, acetonitrile, dimethyl sulfoxide, dimethylacetamide, methyl ethyl ketone, tetrahydrofuran, ethyl acetate, dichloromethane, dimethylformamide, toluene, methanol, ethanol, t-butanol and N,N′-dimethylpropyleneurea.

7. The preparation process as set forth in claim 1, wherein the base is selected from a hydroxide, an oxide, a carbonate, a bicarbonate and an amide of an alkali metal or an alkaline earth metal.

8. The preparation process as set forth in claim 1, wherein extraction with an organic solvent is performed following the treatment with the base and prior to the addition of gentisic acid.

9. The preparation process as set forth in claim 8, wherein the gentisic acid is added directly to the extract obtained by the extraction with the organic solvent or to the concentrate obtained by concentrating the organic solvent of the extraction.

10. The preparation process as set forth in claim 8, wherein the organic solvent used in the extraction is dichloromethane.
